(19) United States
(12) Patent Application Publication
(10) Pub. No.: US 2024/0408256 A1
(43) Pub. Date: Dec. 12, 2024

(54) SYSTEM FOR GENERATING, TRANSPORTING AND APPLYING WATER NANOBUBBLES PLUS OZONE GAS

(71) Applicant: Sergio Hernán LIRA JOANNON, Chimbarongo (CL)

(72) Inventor: Sergio Hernán LIRA JOANNON, Chimbarongo (CL)

(21) Appl. No.: 18/702,463

(22) PCT Filed: Oct. 13, 2022
(86) PCT No.: PCT/CL2022/050103

§ 371 Date: Apr. 18, 2024

(30) Foreign Application Priority Data
Oct. 18, 2021 (CL) 2726-2021

(51) Int. Cl. A61L 2/18; A61L 2/22; A61L 9/14

(52) U.S. Cl. CPC A61L 2/183; A61L 2/22; A61L 9/145; A61L 2202/11; A61L 2202/15; A61L 2209/134; A61L 2209/212

(57) ABSTRACT

A system for generating, transporting and applying water nanobubbles plus ozone gas, as well as active ingredients with a low molecular weight with the aim of simultaneously disinfecting and sanitizing surfaces, people, objects, air and all types of elements in solid, liquid or gaseous state, which allows transporting, applying and screening a volume of air, wherein the water nanobubbles plus ozone gas exhibit a stable and harmless concentration for humans and living beings. Likewise, the system handles a liquid-gas solution, in particular, water nanobubbles plus ozone gas, without the ozone gas molecule being released or fragmented until it strikes a surface, allowing the release and oxidation of the ozone gas on the surface, sanitizing any pathogen and/or virus.

### FIELD OF THE INVENTION

This invention relates to a system for generating, transporting and applying nano bubbles of water plus ozone-gas, as well as low molecular weight active ingredients with the aim of simultaneously disinfecting and sanitizing surfaces, people, objects, air and all types of elements in the solid, liquid or gaseous state.

### BACKGROUND OF THE INVENTION

Today, the systems, structures and methods used for disinfection and sanitization, in particular, given the health emergency, not only seek efficiency and effectiveness to meet their objective, but also seek to reduce and/or eliminate adverse effects on the health of people and living beings, reduce costs and times, eliminate blind spots, in addition to achieving complete sanitization and disinfection simultaneously on surfaces, people, living beings, objects, air and all types of elements.

Likewise, it is especially important that disinfection and sanitation systems are easy to use, modular, mobile, long-range and that they can be applied under any condition, both climatic and with regard to places or spaces that are difficult to access and of different areas of extension. In addition to the above, these systems must comply with health regulations and be supported by rigorous field testing.

In this context, in the systems and structures currently used for the generation, transport and application to disinfect and sanitize, there are solutions that, although they use ozone, given its disinfecting power, they cannot generate, transport and apply nano bubbles with an adequate concentration at distances greater than 10 meters.

In accordance with the foregoing, there is a need in the prior art for a system for generating, transporting and applying nano bubbles of water plus ozone-gas, as well as low molecular weight active ingredients with the aim of simultaneously disinfecting and sanitizing surfaces, people, objects, air and all types of elements in the solid, liquid or gaseous state, improving the effectiveness and efficiency of current disinfection and sanitization systems.

By reviewing and analyzing the prior art, among the closest documents related to the subject matter of the present invention, we can cite patent application CN212327036 in which a disinfection, deodorization and innocuous treatment device based on space analysis and ozone sterilization is described, a document that discloses a technical application other than the invention of the present application, which fails to solve the technical problem of the same.

On the other hand, U.S. Pat. No. 9,764,050 discloses devices and methods that significantly reduce or eliminate germs, bacteria and other microorganisms from objects such as bags, purses, footwear, as well as feet, hands, legs, helmets and other objects, which constitutes a technical solution other than the present invention, which is why it fails to solve the technical problem raised in this invention.

Finally, it is possible to cite as state of the art of the technical field of the present invention, document US2015321228, which discloses an autonomous mobile unit producing foam for cleaning, wherein said unit allows the cleaning and disinfection of agricultural and livestock areas, but does not present the technical means of this invention and does not solve the technical problem addressed by it.

In short, as can be seen in the state of the art previously recited and described, there is no effective solution to the technical problem raised, related to simultaneously disinfecting and sanitizing surfaces, people, objects, air and all kinds of elements in a solid, liquid or gaseous state, which allows to transport, apply and sweep a volume of air, wherein said nano bubbles of water plus ozone-gas present a stable and harmless concentration for people and living beings, likewise the system manages to manipulate a liquid-gas solution, in particular, nano bubbles of water plus ozone-gas, without the ozone-gas molecule being released or fragmented until it impacts on some surface allowing the release and oxidation of the ozone-gas on said surface, achieving the sanitization of all pathogens and/or viruses, the system in addition to applying said nano bubbles on surfaces and/or individuals, performs a sweep that simultaneously displaces the entire mass of air and aerosols in a certain range and in motion, allowing a complete and simultaneous application on individuals, surfaces and environment.

### Objectives and Summary of the Invention

The main objectives of the present invention consist of a system for generating, transporting and applying nano bubbles of water plus ozone-gas, as well as low molecular weight active ingredients the main objectives of the presented with the aim of simultaneously disinfecting and sanitizing surfaces, people, objects, air and all types of elements in a solid, liquid or gaseous state, which allows to transport, apply and sweep a volume of air, wherein said nano bubbles of water plus ozone-gas present a stable and harmless concentration for people and living beings, likewise the system manages to manipulate a liquid-gas solution, in particular, nano bubbles of water plus ozone-gas, without the ozone-gas molecule being released or fragmented until it impacts on some surface allowing the release and oxidation of the ozone-gas on said surface, achieving the sanitization of all pathogens and/or viruses, the system in addition to applying said nano bubbles on surfaces and/or individuals, performs a sweep that simultaneously displaces the entire mass of air and aerosols in a certain range and in motion, allowing a complete and simultaneous application on individuals, surfaces and environment, wherein the system comprises: a) means of transport and traction, terrestrial and/or aquatic (1); b) means of generating ozone-gas in air and/or in liquid (2); c) a voltage inverter (3); and d) means of spraying (4).

### DETAILED DESCRIPTION OF THE INVENTION

The basis of the present invention patent application is a system for generating, transporting and applying nano bubbles of water plus ozone-gas, as well as low molecular weight active ingredients with the aim of simultaneously disinfecting and sanitizing surfaces, people, objects, air and all types of elements in the solid, liquid or gaseous state.

The system of the present invention allows to transport, apply and sweep a volume of air, wherein said nano bubbles of water plus ozone-gas present a stable and harmless concentration for people and living beings, likewise the system manages to manipulate a liquid-gas solution, in particular, nano bubbles of water plus ozone-gas, without the ozone-gas molecule being released or fragmented until it impacts on some surface allowing the release and oxidation of the ozone-gas on said surface, achieving the sanitization of all pathogens and/or viruses.

Furthermore, the system, in addition to applying said nano bubbles on surfaces and/or individuals, performs a sweep that simultaneously displaces the entire mass of air and aerosols in a determined and moving range, allowing a complete and simultaneous application on individuals, surfaces and environment, wherein the system comprises: a) land and/or water means of transport and traction (1); b) means of generating ozone-gas in air and/or in liquid (2); c) a voltage inverter (3); and d) means of spraying (4).

In order to facilitate the understanding of the structural and functional features of the present invention, by way of illustration and not limitation, the following figures are described:

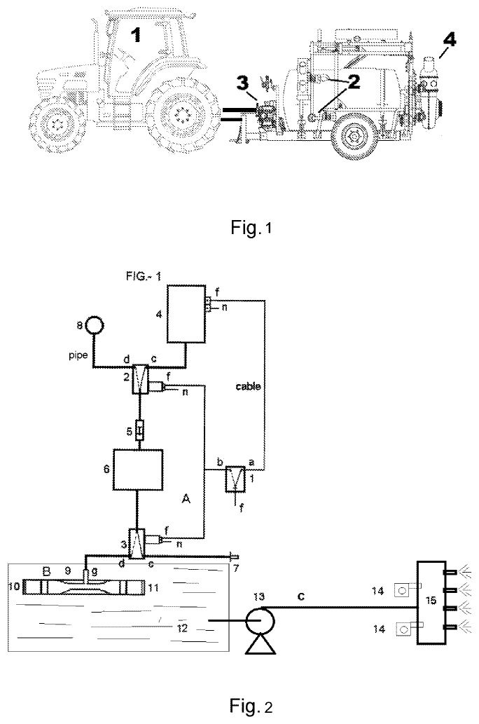

FIG. 1: shows an overview of the system for generating, transporting and applying nano bubbles of water plus ozone-gas, as well as low molecular weight active ingredients with the aim of simultaneously disinfecting and sanitizing surfaces, people, objects, air and all types of elements in the solid, liquid or gaseous state.

FIG. 2: shows a detailed view of the system for generating, transporting and applying nano bubbles of water plus ozone-gas, as well as low molecular weight active ingredients with the aim of simultaneously disinfecting and sanitizing surfaces, people, objects, air and all types of elements in the solid, liquid or gaseous state.

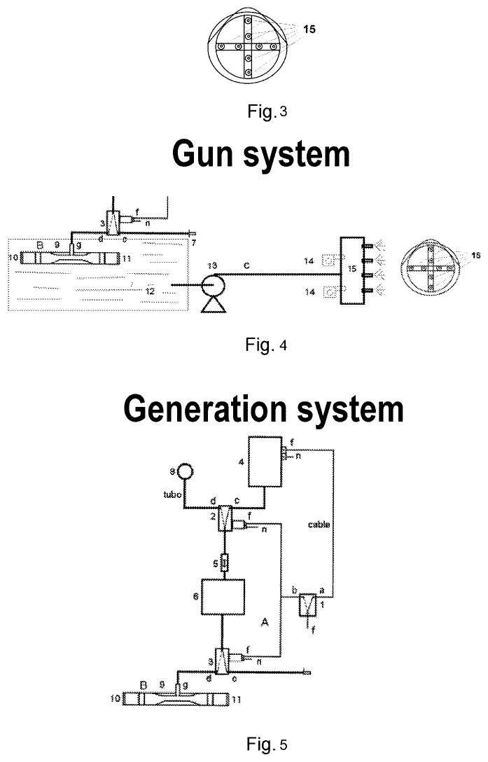

FIG. 3: shows a front view of the spray head of the system of the present invention.

FIG. 4: shows the gun system of the system of the present invention.

FIG. 5: shows the generation system of the system of the present invention.

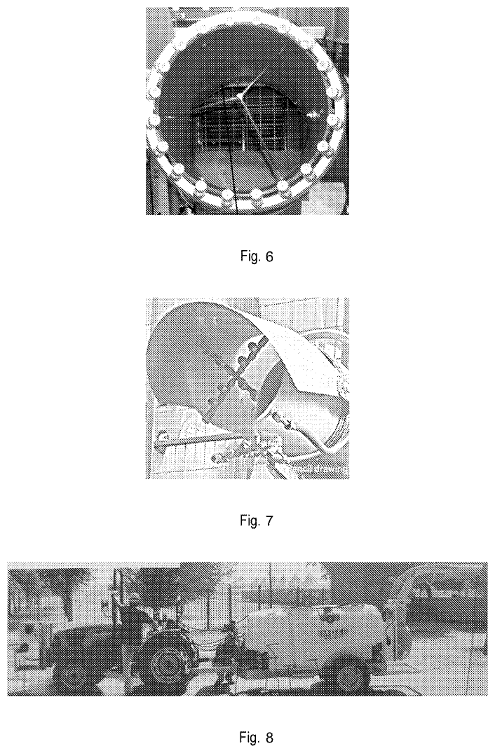

FIG. 6: shows an image of the gun with an inadequate perimeter arrangement of the nozzles.

FIG. 7: shows an image of the gun with a suitable perimeter arrangement of the nozzles.

FIG. 8: shows a photograph of a validation test of the system of the present invention.

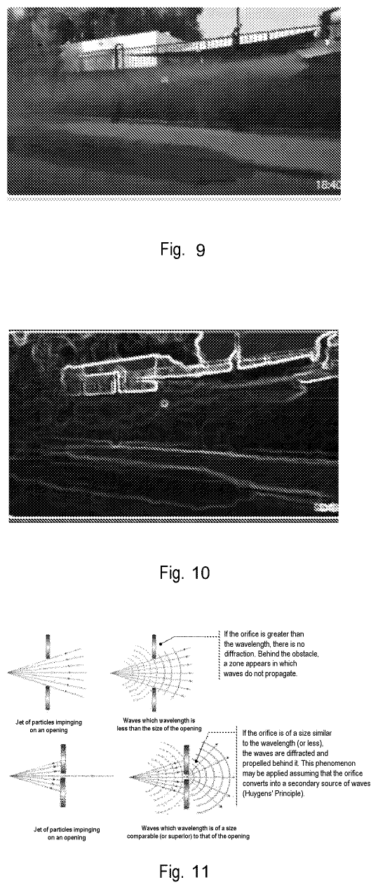

FIG. 9: shows a normal image of a validation test of the system of the present invention.

FIG. 10: shows an image with speed profiles of a validation test of the system of the present invention.

FIG. 11: shows the distribution of the jet of particles impinging on an opening of the system of the present invention.

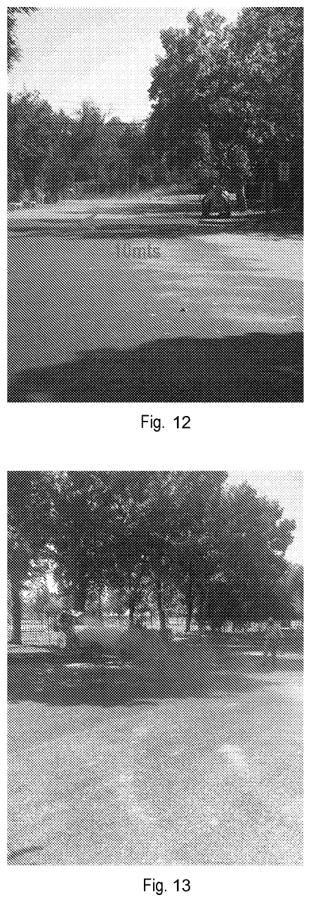

FIG. 12: shows a photograph of a field test of the system of the present invention.

FIG. 13: shows a second photograph of a field test of the system of the present invention.

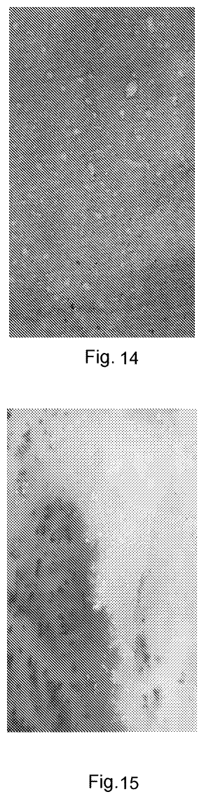

FIG. 14: shows an image of a droplet coverage test at 10 meters of the system of the present invention.

FIG. 15: shows an image of a droplet coverage test at 0.5 meters of the system of the present invention.

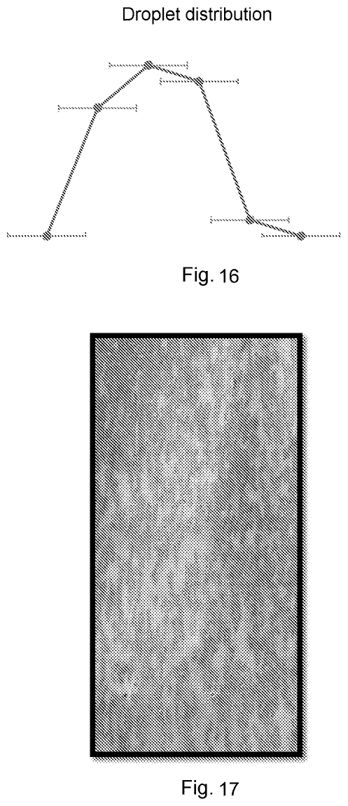

FIG. 16: shows a graph of droplet distribution at 10 meters of the system of the present invention.

FIG. 17: shows a frame of a concentration test of the system of the present invention.

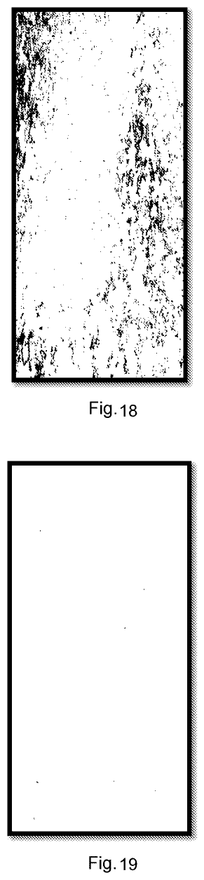

FIG. 18: shows a threshold image of 1.66 ppm of a concentration test of the system of the present invention.

FIG. 19: shows a threshold image of 1.58 ppm of a concentration test of the system of the present invention.

Referring to FIG. 1, it is possible to see the different parts of the system for generating, transporting and applying nano bubbles of water plus ozone-gas, as well as low molecular weight active ingredients is presented with the aim of simultaneously disinfecting and sanitizing surfaces, people, objects, air and all types of elements in a solid, liquid or gaseous state, which allows to transport, apply and sweep a volume of air, wherein said nano bubbles of water plus ozone-gas present a stable and harmless concentration for people and living beings, likewise the system manages to manipulate a liquid-gas solution, in particular, nano bubbles of water plus ozone-gas, without the ozone-gas molecule being released or fragmented until it impacts on some surface allowing the release and oxidation of the ozone-gas on said surface, achieving the sanitization of all pathogens and/or viruses, the system in addition to applying said nano bubbles on surfaces and/or individuals, performs a sweep that simultaneously displaces the entire mass of air and aerosols in a certain range and in motion, allowing a complete and simultaneous application on individuals, surfaces and environment, wherein the system comprises: land and/or water means of transport and traction; means of generating ozone-gas in air and/or in liquid; a voltage inverter; and means of spraying, in particular the following numerical references are disclosed, in a preferred embodiment of the invention:
Reference 1: land and/or water means of transport and traction
Reference 2: means of generating ozone-gas in air and/or in liquid
Reference 3: a voltage inverter
Reference 4: means of spraying

With reference to FIGS. 2, 3, 4 and 5, it is possible to appreciate, in these, a detailed view of the system for generating, transporting and applying nano bubbles of water plus ozone-gas, as well as low molecular weight active ingredients with the aim of simultaneously disinfecting and sanitizing surfaces, people, objects, air and all types of elements in the solid, liquid or gaseous state; a front view of the spray head of the system of the present invention; the gun system of the system of the present invention; and the generation system of the system of the present invention, in particular the following references are disclosed, in a preferred embodiment of the invention:
Reference 1: a circuit breaker
Reference 2: an upper solenoid valve
Reference 3: a lower solenoid valve
Reference 4: a power source
Reference 5: a filter
Reference 6: an ozone generator
Reference 7: a return
Reference 8: one meter pipe
Reference 9: some membranes
Reference 10: a nanobubble generator
Reference 11: a nanobubble generator
Reference 12: a receiving tank
Reference 13: a booster pump
Reference 14: some fans
Reference 15: some nozzles

In preferred embodiments of this system, the system comprises means that allow to transport, apply and sweep a volume by air with said nano bubbles of water plus ozone-gas with a stable and harmless concentration for people and living beings, in an embodiment of the invention, with a height range of up to 3 m and longitudinally up to 50 m; it comprises means for said nano nano bubbles of water plus ozone-gas to be transported and co-assisted by being propelled and co-propelled by being introduced into the center of an air flow; it comprises means for said nano bubbles of water plus ozone-gas, in one embodiment of the invention, to be introduced into the center of an air flow at a velocity of not more than 40 m/s and at a pressure of not more than 10 bar; and land and/or water means of transport and traction, in one embodiment of the invention, correspond to an agricultural tractor not less than 75 HP, with a 12 V to 220 V system, and with a 120 A/h alternator.

In other preferred embodiments, the ozone-gas generating means in air and/or in liquid, correspond to an industrial forced ventilation ozone-gas generating equipment, of 10 g/h of production, at an adjustable concentration of 60 to 80 mg/lt; the ozone-gas generation means in air and/or in liquid, generate a stable concentration of ozone, between 1.57 ppm and 2 ppm; and the voltage inverter is 12 V to 220 V, 3000 W, pure wave.

In other embodiments of the present invention, it is disclosed that the means of spraying comprise: a synthetic or stainless pond for accumulating liquid, with upper lid, vent and lower suction, suspended, entrained or self-propelled; a aspiration filter; a coated membrane or polyamide pump; a command with pressure filter and built-in fibroresin pressure gauge; circuits with stainless steel components and/or synthetic polymer composites; and an air group of the spray gun; whereas the air group of the spray gun comprises: one or more centrifugal fans; and a spray gun, wherein the one or more centrifugal fans are open or closed, and of 15 cm or more in diameter of the turbine and are driven from the power take-off of the means of transport or power source, through cardanic or direct elements to a gearbox of one or two speeds, which expels the air through a directional system called gun at average speeds of 30 m/s or more.

Likewise, in other embodiments of this invention, it is presented that the gun can be manipulated manually, mechanically, electrically, magnetically, electro-pneumatically or hydraulically and/or combining said techniques, to allow 360 degree steering as well as directing it up and down, locating a moving target; the spray gun varies in a range of 100 to 2000 liters being able to be suspended or dragged, with steerable cylinders and hydraulic rotation and comprises one or more hydraulic agitators, one or more titanium diffusers of ozone nano bubbles, a membrane pump of 100 l/min, ceramic nozzles and electrical command; and the mounting arrangement of the nozzles at the outlet of the gun is carried out in the middle of the air flow in one or more sections, in different ways, such as, but not limited to: circular, elliptical, square, rectangular, hexagonal and/or X-shaped, which allow to linearly drive the nano bubbles with ozone-gas molecules without fragmenting them, in the flow rate according to the selected orientation, this non-perimeter arrangement prevents the nano bubbles from colliding with an air flow rate that has a speed and direction different from that propelled by the nozzle, avoiding the fragmentation and friction of the nano bubbles, thus maintaining the ability to encapsulate the required ozone-gas and preventing its release as oxygen, leaving the center of the flow with an adequate concentration of the required mixture.

Finally, in other embodiments of the present invention, the gun comprises means that allow to direct an air flow with said nano bubbles of water plus ozone-gas in a homogeneous stable concentration that allows to sanitize all surfaces, by wetting everything in the selected distance, in particular, it comprises means to wet and displace the air with pathogens in one direction, allowing a complete and simultaneous sanitization in individuals, surfaces and environment; and the gun comprises means that allow sanitizing individuals who transit in the wet section as well as their belongings, clothes and facial environment without having adverse reactions, that is, it comprises means that act on surfaces, air, aerosols and people simultaneously, without adverse effects, in motion and with action bands up to 50 m wide and up to 3 m high.

On the other hand, FIG. 6 shows the image of a gun with a perimeter arrangement of the nozzles, which is not suitable, since the nano bubbles when entering the air flow collide with a stream of air, and are tensioned and fractionated releasing the ozone that they carry encapsulated.

Meanwhile, FIG. 7 shows an image with an arrangement of nozzles within the air flow, which, in any of their forms of positioning, are incorporated into the air flow in the same direction of said flow, wherein the nano bubbles are transported with an output speed from the nozzle and are co-propelled to the air flow, adding speeds and not breaking said nano bubbles.

With respect to FIGS. 8 to 11, FIG. 8 shows a photograph of a validation test of the system of the present invention, while FIGS. 9 and 10 show a normal image of a validation test of the system of the present invention, and an image with velocity profiles of a validation test of the system of the present invention, in the latter it is possible to appreciate that the requirements of fluid mechanics are met, which ratify its scope and distribution of velocities and flow. Subsequently, the effectiveness of ozone distribution on the surface and the environment must be ratified, for which it is necessary to acquire a minimum of 380 g of indigo trisulfonate reagent in salt or solution format with a purity of more than 80%, for the preparation of paper chromatography. The proposed methodology is to check the ozone concentration in the two planes: placing reactive pieces every 50 cm in plane “X” and every 1 meter in plane “Y” giving a total of 90 experimental units per analysis.

FIG. 11: shows the distribution of the jet of particles impinging on an opening of the system of the present invention.

Referring to FIGS. 12 and 13, there are shown photographs of a field test of the system of the present invention. The field test was carried out in the O'Higgins park of the city of Santiago between 11:00 and 13:00 hours, wherein coverage tests and concentration tests were carried out at 50 cm and 10 meters apart from the ground. Both tests were carried out simultaneously with a travel speed of 15 km/h and a pond ozone concentration of 1.75 ppm

Referring to FIGS. 14 to 16, an image of a droplet coverage test at 10 meters of the system of the present invention is shown in FIG. 14, and an image of a droplet coverage test at 0.5 meters of the system of the present invention is seen in FIG. 15. FIG. 16 shows a graph of droplet distribution at 10 meters of the system of the present invention, which delivers a nano bubble distribution test mostly less than 0.02 mm.

Meanwhile, FIGS. 17 to 19 allow to appreciate frames and thresholds in the ozone concentration test, in particular, FIG. 17 shows a frame of a concentration test of the system of the present invention, FIG. 18 shows a threshold image of 1.66 ppm of a concentration test of the system of the present invention, and FIG. 19: shows a threshold image of 1.58 ppm of a concentration test of the system of the present invention.

Said ozone concentration test was developed on reagent paper chromatography and then measured its concentration through a digital colorimetric curve of CieLab coordinates 1.66 ppm which corresponds to a CIELAB coordinate (48.86 6.90 −45.45), and 1.58 ppm (41.37 7.86 −44.64), which as shown in FIG. 16, 92% of the surface has a concentration greater than 1.66 ppm and if we consider the threshold of 1.58 ppm we would have 99.9% of the surface, as shown in FIG. 19.

Both coverage and concentration tests have shown to be efficient in every aspect in the critical range of 10 meters, to improve coverage you can lower the speed of travel and increase the power to increase from 10 to 20 meters, but in general all tests show a concentration greater than 1.58 ppm of ozone on the reactive surface, the droplet size is mostly less than 0.02 mm, which guarantees a greater and active unit coverage.

After the detailed description of the present invention in its various embodiments, in the next section the set of claims is presented, with the technical detail associated with the described system.

1. A system for generating, transporting and applying nano bubbles of water plus ozone-gas, as well as low molecular weight active ingredients with the aim of simultaneously disinfecting and sanitizing surfaces, people, objects, air and all types of elements in a solid, liquid or gaseous state, which allows to transport, apply and sweep a volume of air, wherein said nano bubbles of water plus ozone-gas present a stable and harmless concentration for people and living beings, likewise the system manages to manipulate a liquid-gas solution, in particular, nano bubbles of water plus ozone-gas, without the ozone-gas molecule being released or fragmented until it impacts on some surface allowing the release and oxidation of the ozone-gas on said surface, achieving the sanitization of all pathogens and/or viruses, the system in addition to applying said nano bubbles on surfaces and/or individuals, performs a sweep that simultaneously displaces the entire mass of air and aerosols in a certain range and in motion, allowing a complete and simultaneous application on individuals, surfaces and environment, wherein the system comprises:
a) land and/or water means of transport and traction (1);
b) means of generating ozone-gas in air and/or in liquid (2);
c) a voltage inverter (3); and
d) means of spraying (4).

2. The system for generating, transporting and applying nano bubbles of water plus ozone-gas according to claim 1, wherein it comprises means that allow to transport, apply and sweep a volume by air with said nano bubbles of water plus ozone-gas with a stable and harmless concentration for people and living beings, in an embodiment of the invention, with a height range of up to 3 m and longitudinally up to 50 m.

3. The system for generating, transporting and applying nano bubbles of water plus ozone-gas according to claim 1, wherein the system comprises means for said nano bubbles of water plus ozone-gas to be transported and co-assisted by being propelled and co-propelled by being introduced into the center of an air flow.

4. The system for generating, transporting and applying nano bubbles of water plus ozone-gas according to the preceding claim, wherein the system comprises means for said nano bubbles of water plus ozone-gas, in one embodiment of the invention, to be introduced into the center of an air flow at a velocity of not more than 40 m/s and at a pressure of not more than 10 bar.

5. The system for generating, transporting and applying nano bubbles of water plus ozone-gas according to claim 1, wherein the means of land and/or water transport and traction, in one embodiment of the invention, correspond to an agricultural tractor not less than 75 HP, with a system from 12 V to 220 V, and with a 120 A/h alternator.

6. The system for generating, transporting and applying nano bubbles of water plus ozone-gas according to claim 1, wherein the means for generating ozone-gas in air and/or in liquid, in one embodiment of the invention, correspond to an industrial forced ventilation ozone-gas generating equipment, of 10 g/h of production, at a controllable concentration of 60 to 80 mg/l.

7. The system for generating, transporting and applying nano bubbles of water plus ozone-gas according to the preceding claim, wherein the means for generating ozone-gas in air and/or in liquid, in an embodiment of the invention generate a stable concentration of ozone, between 1.57 ppm and 2 ppm.

8. The system for generating, transporting and applying nano bubbles of water plus ozone-gas according to claim 1, wherein in one embodiment of the invention the voltage inverter is 12 V to 220 V, 3000 W, pure wave.

9. The system for generating, transporting and applying nano bubbles of water plus ozone-gas according to claim 1, wherein in one embodiment of the invention the means of spraying comprises:
i. a synthetic or stainless pond for accumulating liquid, with upper lid, vent and lower suction, suspended, entrained or self-propelled;
ii. a aspiration filter;
iii. a coated membrane or polyamide pump;
iv. a command with pressure filter and built-in fibroresin pressure gauge;
V. circuits with stainless steel components and/or synthetic polymer composites; and
vi. an air group of the spray gun.

10. The system for generating, transporting and applying nano bubbles of water plus ozone-gas according to the preceding claim, wherein the air group of the spray gun comprises:
i. one or more centrifugal fans; and
ii. a spray gun.

11. The system for generating, transporting and applying nano bubbles of water plus ozone-gas according to the preceding claim, wherein in an embodiment of the invention, the one or more centrifugal fans are open or closed, and of 15 cm or more in diameter of the turbine.

12. The system for generating, transporting and applying nano bubbles of water plus ozone-gas according to the preceding claim, wherein in an embodiment of the invention, the one or more centrifugal fans are driven from the power take-off of the means of transport or power source, through cardanic or direct elements to a gearbox of one or two speeds, which expels the air through a directional system called a gun at average speeds of 30 m/s or more.

13. The system for generating, transporting and applying nano bubbles of water plus ozone-gas according to the preceding claim, wherein in an embodiment of the invention, the gun can be manipulated manually, mechanically, electrically, magnetically, electro-pneumatically or hydraulically and/or combined said techniques, to allow a 360-degree targeting as well as targeting it up and down, locating a moving target.

14. The system for generating, transporting and applying nano bubbles of water plus ozone-gas according to claim 10, wherein in an embodiment of the invention the spray gun varies in a range of 100 to 2000 liters and can be suspended or dragged, with steerable cylinders and hydraulic rotation.

15. The system for generating, transporting and applying nano bubbles of water plus ozone-gas according to the preceding claim, wherein the spray gun comprises one or more hydraulic agitators, one or more titanium diffusers of nano bubbles of ozone, a membrane pump of 100 l/min, ceramic nozzles and electrical command.

16. The system for generating, transporting and applying nano bubbles of water plus ozone-gas according to the preceding claim, wherein in an embodiment of the invention, the mounting arrangement of the nozzles at the exit of the gun is carried out in the middle of the air flow in one or more sections, in different ways, such as for example, but not limited to: circular, elliptical, square, rectangular, hexagonal and/or X-shaped, which allow to linearly drive the nano bubbles with ozone-gas molecules without fragmenting them, in the flow rate according to the selected orientation, this non-perimeter arrangement prevents the nano bubbles from colliding with an air flow rate that has a speed and direction different from that propelled by the nozzle, avoiding the fragmentation and friction of the nano bubbles, thus maintaining the ability to encapsulate the required ozone-gas and preventing its release as oxygen, leaving the center of the flow with an adequate concentration of the required mixture.

17. The system for generating, transporting and applying nano bubbles of water plus ozone-gas according to the preceding claim, wherein in one embodiment of the invention, the gun comprises means that allow to direct an air flow with said nano bubbles of water plus ozone-gas in a homogeneous stable concentration that allows to sanitize all surfaces, by wetting everything in the selected distance, in particular, it comprises means to wet and displace the air with pathogens in one direction, allowing a complete and simultaneous sanitization in individuals, surfaces and the environment.

18. The system for generating, transporting and applying nano bubbles of water plus ozone-gas according to the previous claim, wherein the gun comprises means that allow to sanitize the individuals that transit in the wet section as well as their belongings, clothes and facial environment without having adverse reactions, that is, it comprises means that act on surfaces, air, aerosols and people simultaneously, without adverse effects, in movement and with action bands of up to 50 m wide and up to 3 m high.
